(19) United States
(12) Patent Application Publication
(10) Pub. No.: US 2017/0059567 A1
(43) Pub. Date: Mar. 2, 2017

(54) METHODS AND SYSTEMS FOR ELECTROCHEMICALLY DETECTING OR QUANTIFYING AN ANALYTE

(71) Applicant: THE UNIVERSITY OF UTAH RESEARCH FOUNDATION, Salt Lake City, UT (US)

(72) Inventors: Himanshu Jayant Sant, Salt Lake City, UT (US); Bruce K. Gale, Salt Lake City, UT (US); Harikrishnan Jayamohan, Salt Lake City, UT (US); Christopher J. Lambert, Salt Lake City, UT (US); John Minson, South Jordan, UT (US)

(21) Appl. No.: 15/027,405

(22) PCT Filed: Oct. 7, 2014
(86) PCT No.: PCT/US14/59537

§ 371 Date: Apr. 5, 2016

Related U.S. Application Data
(60) Provisional application No. 61/961,186, filed on Oct. 7, 2013.

(51) Int. Cl. G01N 33/569; G01N 33/543; C12Q 1/68

(52) U.S. Cl. CPC G01N 33/56916; C12Q 1/6825; G01N 33/54333; G01N 33/5438; G01N 2333/245; G01N 2458/10

(57) ABSTRACT

Methods of electrochemically detecting or quantifying an analyte by coupling a plurality of redox-active agents (e.g., guanine-rich oligonucleotides) to the analyte are disclosed. More particularly, this application discloses affinity-based methods for isolating one or more analytes from a sample and subsequently detecting or determining the concentration of the one or more analytes. Detecting or determining the concentration of one or more analytes may involve measuring the extent of oxidation of guanine nucleobases that have been or are coupled to the analyte.

### RELATED APPLICATIONS

This application claims priority to U.S. Provisional Application No. 61/961,186, filed on Oct. 7, 2013, titled AUTOMATED PATHOGEN DETECTION SYSTEM, which is hereby incorporated by reference in its entirety.

### TECHNICAL FIELD

This application generally relates to detection methods and systems, and more particularly to methods of electrochemically detecting and/or quantifying an analyte.

### BACKGROUND

Electrochemical detection is a relatively easy, rapid, and inexpensive technique for detecting and quantifying analytes. Many glucose meters, for example, use electrochemical detection to assess glucose levels in a blood sample. Despite its many advantages, however, electrochemical detection has generally been limited to the detection of a relatively small number of analytes that both (1) have suitable redox properties and (2) are present in high enough concentrations to be detected by an electrochemical sensor. Because many analytes—including many diagnostically useful analytes—lack suitable redox properties and/or are generally not found in test samples in a high enough concentration to facilitate direct electrochemical detection, there is a need for methods and systems that facilitate the electrochemical detection and/or quantification of a wider variety (and concentration) of analytes.

Diagnostically useful analytes that may be difficult to detect or quantify via electrochemical means may include proteins (e.g., surface proteins on bacteria and viruses, protein toxins, enzymes, immunoglobulins), nucleic acids, small molecules, hormones, and/or many other analytes. One class of analytes that may be particularly difficult to detect or measure using conventional electrochemical detection techniques is bacteria, such as, for example, E. coli, especially when the bacteria is found at low concentrations in the relevant sample.

Although most strains of E. coli are generally harmless, the detection of E. coli (and more particularly, pathogenic serotypes of E. coli, such as O157:H7) is of significant importance. For example, the presence of E. coli is often used to assess whether a sample is contaminated with fecal matter, as E. coli is excreted from the lower intestine of many warm-blooded organisms.

Additionally, the detection of particular pathogenic bacterial strains, such as E. coli O157:H7, is of particular importance as such bacteria are a major source of food- and water-borne diseases throughout the world. Yang & Li, Simultaneous Detection of Escherichia Coli 0157H7 and Salmonella Typhimurium using Quantum Dots as Fluorescence Labels, 131 Analyst 394-401 (2006). According to the World Health Organization, gastrointestinal infections (which are often caused by pathogenic bacteria) kill about 2.2 million people globally each year. WHO, Water-Related Diseases (2014), available at http://www.who.int/water_sanitation_health/diseases/diarrhoea/en/. Ideally, methods of detecting E. coli and other analytes are very sensitive, as even very small amounts of some strains of E. coli (10-100 viable organisms) can cause human infection. Yang & Li, 131 Analyst 394-401.

Current methods for detecting E. coli, such as membrane filtration, plate counting, turbidimetry, and multiple tube fermentation may be time-consuming, complex, and/or require specially trained personnel. Additionally, these techniques are typically not suitable for use in the field, which is a significant limitation given the need to monitor pathogenic bacteria in geographically remote locations. Thus, improved methods, systems, and tools for detecting and/or quantifying an analyte are needed.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a first schematic diagram illustrating a process for detecting an analyte via differential pulse voltammetry.

FIG. 2 is a second schematic diagram illustrating a process for detecting an analyte via differential pulse voltammetry.

FIG. 3 is a schematic diagram illustrating steps for preparing an electrode for detection of an analyte.

FIG. 4 is a graph depicting cyclic voltammetry data for the electrodeposition of graphene oxide on a glassy carbon electrode surface.

FIG. 5 provides fluorescence images of samples processed by methods described herein.

FIG. 6 is a graph depicting absolute differential pulse voltammetry signals detected from samples with differing initial concentrations of E. coli O157:H7.

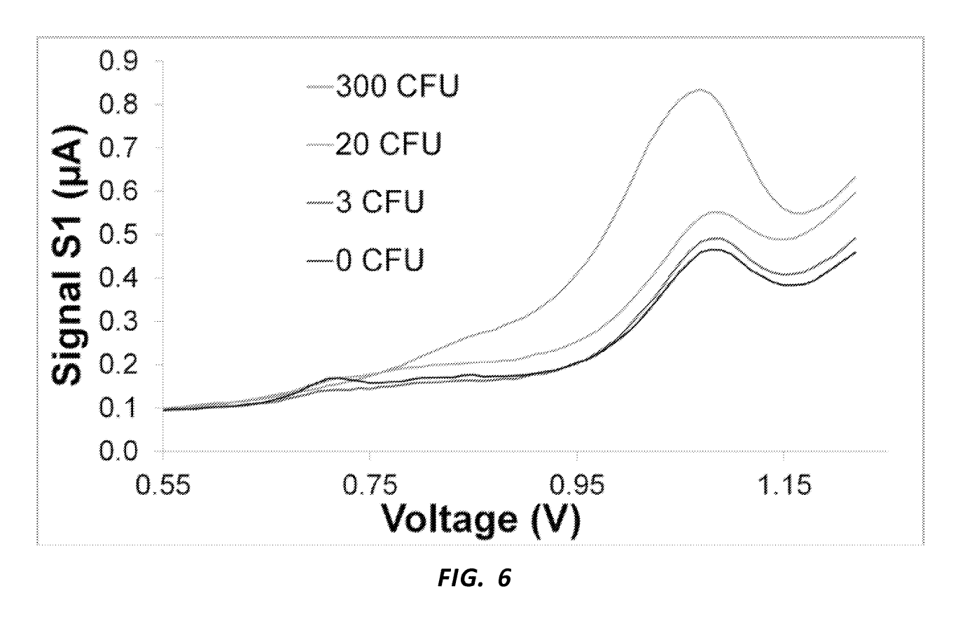

FIG. 7 is a graph depicting the change in differential pulse voltammetry signals as a function of the number of colony-forming units in a sample.

FIG. 8 is a bar graph depicting the change in differential pulse voltammetry signals for four different water samples.

FIG. 9 is a graph depicting the change in differential pulse voltammetry signals as a function of the concentration of analyte in a sample.

### DETAILED DESCRIPTION

This disclosure is related to methods and systems for detecting and/or determining the concentration of one or more analytes via electrochemical means. It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order of specific steps or actions may be modified.

As used herein, the term “sequence,” when used with reference to an oligonucleotide, refers to a sequence of the oligonucleotide that comprises ten or more oligonucleotides. Two oligonucleotides are complementary to each other if, when they are aligned antiparallel to each other, the nucleotide bases at each position are appropriately paired (e.g., A-T, G-C). Two oligonucleotides are substantially complementary to each other if, when they are aligned antiparallel to each other, more than 85% of the nucleotide bases at each position are appropriately paired. The term “sample” refers not only to an initial sample to be processed, but also to the portion of that initial sample that retains the analyte as the sample is processed.

The methods and systems described below may permit the detection of a greater variety of analytes via electrochemical means and/or decrease the amount of analyte that is needed for electrochemical detection. More particularly, the methods and systems described below may allow for the indirect electrochemical detection of a wide variety of analytes by (1) binding a plurality of redox-active agents to an analyte, and (2) detecting and/or quantifying the extent of oxidation of the redox-activate agents that had bound to the analyte. In some circumstances, the dynamic range of the methods or systems described herein may be manipulated by appending a large number of redox-active moieties to a single analyte of interest. Some methods disclosed herein for detecting analytes may be carried out in relatively short amounts of time, such as less than about 2, 3, or 4 hours. The length of time required to carry out the disclosed methods may vary based on the particularities of each method and the characteristics of the analyte to be detected. Additionally, some methods may allow for the detection of multiple analytes simultaneously or nearly simultaneously. In other words, some methods and systems may permit multiplexing.

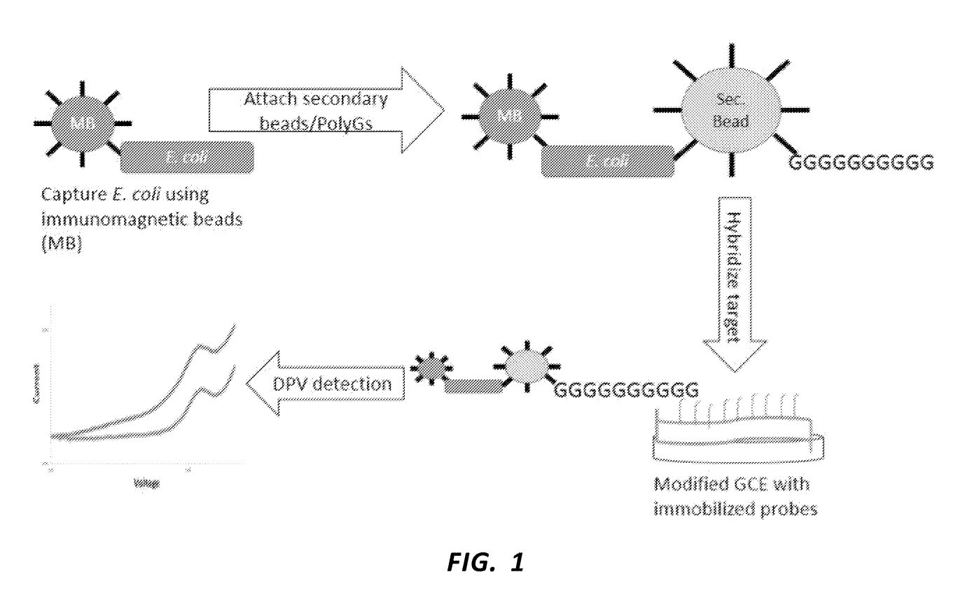

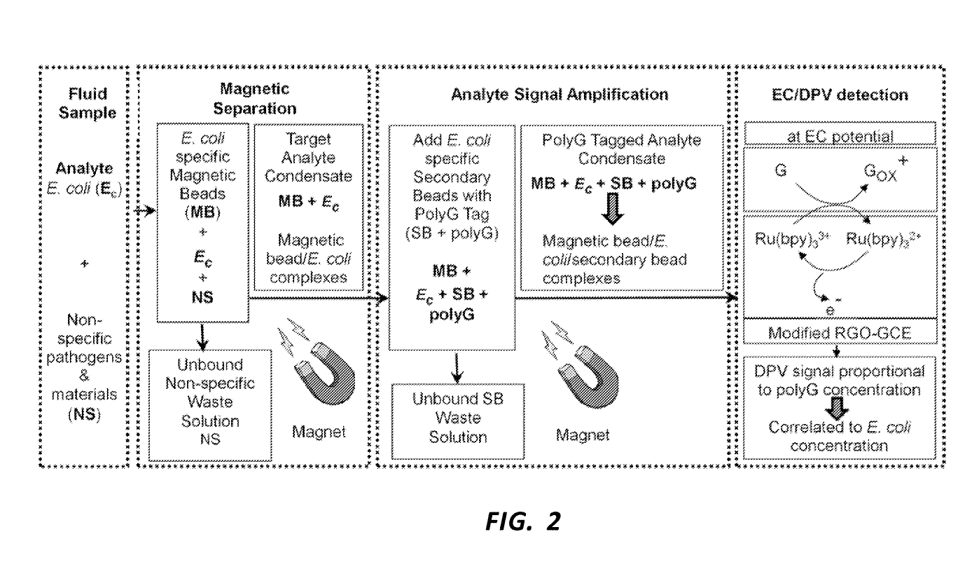

FIGS. 1 and 2 provide graphical depictions of some steps and components that may be used in connection with the methods, techniques, and systems described herein. Briefly, an analyte (such as, without limitation, E. coli) in a sample may be separated from other components in the sample by immunoseparation. In some embodiments, such immunoseparation is accomplished through the use of a first analyte-binding agent that comprises a magnetic bead. A second analyte-binding agent comprising (1) a non-magnetic bead, (2) an analyte-binding portion, and (3) a plurality of redox-active molecules (e.g., guanine-rich (“G-rich”) oligonucleotides) may also be allowed to bind to the analyte. The binding of the second analyte-binding agent to the analyte may couple any suitable number of guanine nucleobases to the analyte. For example, the binding of the second analyte-binding agent to the analyte may couple more than about 10, 10², 10³, 10⁴, 10⁵, 10⁶, 10⁸, 10¹⁰, 10¹², or 10¹⁵ nucleobases to the analyte. After removing any excess second analyte-binding agent, the G-rich oligonucleotides of the remaining second analyte-binding agent may be hybridized to cytosine-rich (“C-rich”) oligonucleotides that are immobilized, in some instances, to a substrate, such as an electrode. In some embodiments, the electrode comprises a glassy carbon electrode or a silicon wafer upon which graphene oxide has been deposited. With the G-rich oligonucleotides hybridized to the C-rich oligonucleotides in this manner, the guanine nucleobases of the G-rich oligonucleotides may be oxidized and detected electrochemically (e.g., by differential pulse voltammetry). The observed signal may be used to estimate or determine the concentration of analyte within the sample.

The steps and components shown in FIGS. 1 and 2, along with other steps and components of the methods and systems described herein, are set forth in additional detail below.

In some embodiments, a sample may be concentrated and/or diluted prior to processing in a manner similar to that shown in FIGS. 1 and 2. For example, a sample may be concentrated by forcing a portion of the sample through a filter with pore sizes smaller than the analyte to be detected. More particularly, in some embodiments, the liquid portion of a sample may pass through the filter, leaving a retentate on the filter that may be resuspended in a relatively small container. Concentration of the sample may lower the costs of conducting an assay. For example, concentration of the sample may reduce the number of antibody-appended magnetic beads required to immunoseparate an analyte in a mixture.

In some embodiments, an analyte within a sample (whether concentrated or not) may be isolated, enriched, and/or immobilized through the use of a first analyte-binding agent. For example, a first analyte-binding agent comprising a particle (such as, without limitation, a magnetic bead) and an analyte-binding region (such as, without limitation, a monoclonal or polyclonal antibody, hapten, peptide, protein, aptamer, DNA, RNA, etc.) may initially be mixed with a sample that includes an analyte. The analyte-binding region of the first analyte-binding agent may then bind to the analyte in a covalent or non-covalent fashion to form a first complex. An analyte-rich portion of the sample may then be spatially separated from an analyte-depleted portion of the sample by any suitable means, such as by placing the sample in a magnetic field. The analyte-depleted portion of the sample may then be removed. For example, the analyte-depleted portion disposed adjacent an analyte-rich portion of the sample may be removed by pipette or decantation. In some embodiments, the remaining analyte-rich portion may be resuspended by mixing the analyte-rich portion with a buffer or other solution and then using a magnetic field to generate another analyte-depleted portion that may be removed from the analyte-rich portion. The process of separating analyte-rich and analyte-depleted portions from one another and subsequently removing the analyte-depleted portion may optionally be repeated until the analyte has been purified to a desired or sufficient extent. Purification of the analyte from surrounding material may remove materials that could interfere with the detection and/or quantification process.

A second analyte-binding agent may also be added to a vessel (e.g., a tube) that contains the analyte (or the analyte-rich portion). The second analyte-binding agent may be added prior to, concurrent with, or after the addition of the first analyte-binding agent. The second analyte-binding agent may comprise a first portion and a second portion. The first portion of the second analyte-binding agent (e.g., an antibody, hapten, protein, aptamer, etc.), may be configured to bind to the analyte. In some embodiments, the second analyte-binding agent may bind to a region of the analyte that differs from the region of the analyte to which the first analyte-binding agent binds. For example, the first analyte-binding agent may comprise a first antibody with specificity to a first epitope of the analyte, while the second analyte-binding agent comprises a second antibody that binds preferentially to a second epitope of the analyte. The use of a second antibody that binds preferentially to a second epitope may be advantageous as few molecules, other than the analyte of interest, are likely to bind to two different antibodies that are specific to different epitopes. In other words, the use of a second analyte-binding agent that binds preferentially to a second epitope of the analyte may increase the specificity of detection. In some embodiments, the first analyte-binding agent and the second analyte-binding agent bind to the same region of the analyte. In such embodiments, the second analyte-binding agent may substantially displace the first analyte-binding agent due to its increased concentration relative to the first analyte-binding agent and/or its increased affinity (e.g., lower K_{d}) to the region of the analyte.

The second portion of the second analyte-binding agent may comprise a first plurality of redox-active agents, such as oligonucleotides comprising one or more guanine nucleobases. For example, the second portion of the second analyte-binding agent may be a first plurality of oligonucleotides that are appended to a non-magnetic bead. In some embodiments, the first plurality of oligonucleotides comprises one or more guanine nucleobases. For example, the first plurality of oligonucleotides may have a plurality of nucleobases, the majority of which are guanine nucleobases. In further embodiments, the plurality of nucleobases are at least 60%, 70%, 80%, 90%, and/or 100% guanine. In other embodiments, the nucleobases of the plurality of nucleobases are less than 50% guanine nucleobases. In some embodiments, the G-rich oligonucleotides do not comprise cytosine nucleobases.

The second analyte-binding agent may further comprise a non-magnetic bead (e.g., a styrene, polystyrene, porous polystyrene, polymeric, agarose, glass, ceramic, or composite bead). In other or further embodiments, the non-magnetic bead (and the second analyte binding agent in general) may not comprise a gold particle. The use of a second analyte binding agent that lacks gold may facilitate electrochemical detection. For example, the second analyte-binding agent may comprise a non-magnetic streptavidin-coated polystyrene bead, an antibody with specificity for the analyte of interest that is conjugated to the streptavidin-coated polystyrene bead, and a plurality of biotinylated oligonucleotides that bind to the streptavidin of the streptavidin-coated bead. In some embodiments, a particle (e.g., a non-magnetic bead) of the second analyte-binding agent may be roughly spherical and have a diameter of between 1 and 400 microns. In other or further embodiments, the surface of the non-magnetic bead or particle may be smooth, rough, or porous. Additionally, in some embodiments, the non-magnetic bead is attached to other particles or beads. The size, shape, and surface characteristics of the bead may be altered to accommodate the desired number and/or orientation of appended oligonucleotides. For example, in some embodiments the bead is hollow or donut-shaped.

In some second analyte-binding agents that comprise a bead, the first portion of the second analyte-binding agent (which is configured to bind to the analyte) and the first plurality of oligonucleotides may be disposed in any suitable manner with respect to the bead. For example, in some embodiments, the first portion (i.e., the analyte binding portion) and/or the second portion (comprising a plurality of redox-active agents such as guanine-rich oligonucleotides) may be disposed (e.g., immobilized) on an interior surface of a bead. In other words, the first and/or second portion of the second analyte-binding agent may be disposed within a hollow portion of the bead.

Upon addition of the second analyte-binding agent to a sample comprising the analyte, the second analyte-binding agent may bind to the analyte. Excess second analyte-binding agent that does not bind to the analyte may then be removed in a manner similar to that described above. For example, the sample may be placed in a magnetic field to draw the first analyte-binding agent (due to the magnetic bead of the first analyte-binding agent) and the rest of the complex of which it is a part (comprising the analyte and the second analyte-binding agent) away from other components of the mixture. The remaining portion (i.e., an analyte-depleted portion comprising excess second analyte-binding agent that does not bind to the analyte) may then be removed in any suitable manner (e.g., by pipette, decantation, microfluidics).

The number of oligonucleotides per second analyte-binding agent may be adjusted to increase or decrease the detection limit and/or dynamic range of a method or system for detecting and/or quantifying an analyte, as preferred by the desired application. For example, increasing the number of oligonucleotides that are bound to each of the second analyte-binding agents may increase the magnitude of the electrochemical signal that is eventually generated. For example, the number of oligonucleotides per second analyte-binding agent (e.g., the number of oligonucleotides that are attached to the streptavidin-coated polystyrene bead) can be anywhere between 10²-10¹³, 10⁵-10¹³, 10¹⁰-10¹³, or 10⁷-10¹². In some embodiments, the number of oligonucleotides that are attached to the streptavidin-coated polystyrene bead exceeds 10¹³.

In some embodiments, the number of guanine nucleobases per oligonucleotide may be adjusted to increase the detection limit and/or dynamic range of a method or system for detecting and/or quantifying an analyte. Because guanine nucleobases are the primary reductant of DNA under typical conditions, the non-background signal observed in differential pulse voltammetry experiments is primary attributable to the oxidation of guanine nucleobases. Thus, by increasing the number of guanine nucleobases per oligonucleotide, the generated electrochemical signal may be increased and the detection limit of the analyte may be lowered. The number of guanine nucleobases per oligonucleotide can be anywhere between 10 and 400 nucleobases.

After excess second analyte-binding agent has been removed from the sample, the first plurality of oligonucleotides (e.g., G-rich oligonucleotides) that are or had been coupled to the analyte may then be added to an electrode which has been functionalized with a complementary or substantially complementary second plurality of oligonucleotides. The first plurality of oligonucleotides may be added to the electrode alone, or in combination with other components of the sample. For example, in some embodiments, the first plurality of oligonucleotides is coupled to one or more of (1) the remaining portions of a second analyte-binding agent, (2) the analyte, and (3) the first analyte-binding agent when added to the surface of the electrode. In some embodiments, the first plurality of oligonucleotides may be separated from one or more of the above-referenced components prior to addition to the electrode surface. For example, in some embodiments, the second analyte-binding agent may comprise a labile linkage that may be selectively cleaved to enable separation of the first plurality of oligonucleotides from other components in a mixture. In other embodiments, the first plurality of oligonucleotides may be eluted from other components in the mixture by some other method.

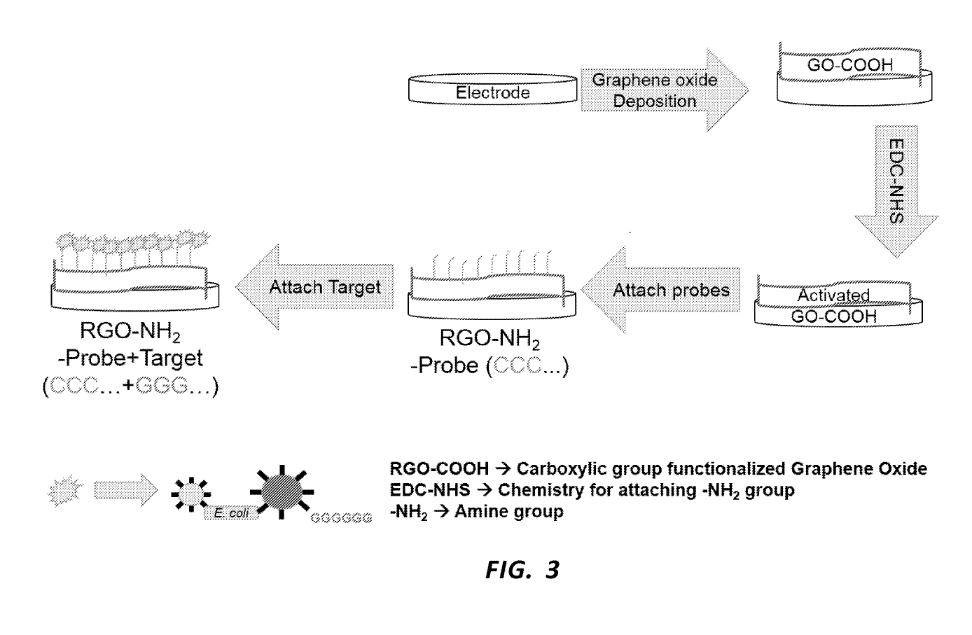

The functionalized electrode upon which the second plurality of oligonucleotides is immobilized may comprise a glassy carbon electrode or a silicon wafer on which silicon dioxide has been deposited. A representative process for preparing an electrode for detection of an analyte via differential pulse voltammetry is shown in FIG. 3. As illustrated in FIG. 3, graphene oxide may be deposited on an electrode in any suitable manner, such as that reported in Chen et al., Direct Electrode position of Reduced Graphene Oxide on Glassy Carbon Electrode and its Electrochemical Application, 13 Electrochemistry Communications 133-37 (2011), which is hereby incorporated by reference in its entirety. After deposition of the graphene oxide, the electrode may be activated to facilitate conjugation of a second plurality of oligonucleotides. For example, the electrode may be functionalized to create carboxylic acid functional groups on the electrodeposited graphene. More particularly, an electrode to which graphene oxide has been deposited may be etched with NaOH to create carboxylic acid functional groups. In other embodiments, carboxylic acid-functionalized graphene oxide is directly deposited on the electrode.

The carboxyl groups may be converted to amine-reactive N-hydroxysuccinimide esters by reaction with sulfo-N-hydroxysuccinimide. Subsequently, a second plurality of oligonucleotides (e.g., amine-terminated C-rich oligonucleotides) may be immobilized to the surface of the electrode by reacting such oligonucleotides with N-hydroxysuccinimide esters on the surface of the electrode. In some embodiments, the second plurality of oligonucleotides does not include a guanine nucleobase. After the second plurality of oligonucleotides has been immobilized on the electrode and the electrode surface has been washed, the electrode surface is prepared for hybridization of the first plurality of oligonucleotides to the second plurality of oligonucleotides.

In some embodiments, the number of immobilized oligonucleotides on the electrode may be adjusted (i.e., increased or decreased) to affect the detection limit and/or dynamic range of a method or system for detecting and/or quantifying an analyte. For example, the surface area of the electrode may be increased and/or the density of the oligonucleotides that are immobilized on the electrode may be increased, thereby increasing the dynamic range of the electrode. Conversely, if needed or desired, the number of immobilized oligonucleotides on the electrode may be decreased by decreasing the surface area of the electrode and/or the density of the oligonucleotides that are immobilized to the electrode. The dynamic range of methods and systems for detecting analytes may be tuned in other ways as well. For example, in addition to increasing the number of guanine molecules that are coupled to the analyte of interest (as noted above), the dynamic range may also be increased by serially diluting and/or concentrating the original sample. In some embodiments, the diluted and/or concentrated sample may be assigned a unique working electrode and a unique set of complementary oligonucleotide tags and probes.

After the electrode surface has been functionalized by immobilizing the second plurality of oligonucleotides to its surface, the first plurality of oligonucleotides may be added to the electrode surface, thereby allowing the first plurality of oligonucleotides to bind (e.g., hybridize) to the immobilized second plurality of oligonucleotides. The binding of the first plurality of oligonucleotides to a second plurality of oligonucleotides that are near the working electrode may generate a larger electrochemical signal than if the redox-active molecules were distributed randomly throughout the solution.

After the first plurality of oligonucleotides has hybridized with the second plurality of oligonucleotides that are immobilized on the electrode, amperometry (e.g., differential pulse voltammetry) may be used to oxidize the guanine nucleobases (guanine→guanine⁺+e⁻) of a first plurality of oligonucleotides and to determine the extent of such oxidation. The extent of such oxidation generally correlates with the amount of analyte in the initial sample.

The detection and/or quantification of a redox reaction (e.g., oxidation of guanine nucleobases) may be mediated by a mediator, such as a redox-active species in solution with about the same redox potential as the oxidant (e.g., guanine; 1.04 V). The mediator may transport electrons from the hybridized probe-target oligonucleotides to the sensor surface. In some embodiments, the mediator is a metal complex in solution, such as aqueous Ru(bpy)₃²⁺ and/or Ru(bpy)₃³⁺.

The use of a mediator, such as Ru(bpy)₃²⁺ and/or Ru(bpy)₃³⁺, may have advantages over some processes that lack such a mediator. For example, the signal generated by oxidation of guanine in the presence of Ru(bpy)₃²⁺ and/or Ru(bpy)₃³⁺ may be larger than the signal generated when guanine is oxidized in the absence of Ru(bpy)₃²⁺ and/or Ru(bpy)₃³⁺. This increased signal may facilitate the detection and/or measurement of low analyte levels.

The mediator may also facilitate the determination of baseline noise levels. For example, when there is no guanine oxidation during a scan, repeated measurements of the oxidation and reduction signals associated with the mediator should, in theory, be identical. Thus, any variation in repeated scans may be identified as noise. This variation may be used to inform the decision about whether a sample includes a particular analyte. Ideally, the mediator signal is relatively constant from scan to scan, from working electrode to working electrode, and from sensor to sensor. Thus, the mediator may be used (1) as a control to demonstrate that the sensor is working, and/or (2) as a calibrant to compare analyte signals from different sensors.

In one embodiment, an operator may detect and/or quantify the extent of oxidation by first performing a first amperometric detection scan to produce a first signal. Generally, the first signal includes a signal from guanine oxidation and any background noise. This first scan oxidizes essentially all of the guanine nucleobases in the first plurality of oligonucleotides. In an embodiment that uses Ru(bpy)₃²⁺ and/or Ru(bpy)₃³⁺ as a mediator, the following reactions may occur during the first scan:

Ru(bpy)₃²⁺→Ru(bpy)₃³⁺+e⁻

Ru(bpy)₃³⁺+guanine→Ru(bpy)₃²⁺+guanine⁺

The operator may then perform a second scan. Since guanine⁺ is resistant to reduction back to neutral guanine, the signal from this second scan may include essentially only background noise. The operator may then perform a third scan. The third scan, which also may include only background noise, may be compared with the second scan to assess the variability of background noise. From this variability, an appropriate cut-off point for determining the presence or absence of an analyte may be selected. For example, an analyte may be considered present only if the generated signal from an unknown sample is greater than the selected cut-off value (e.g., the greatest variation in signal between the second scan and the third scan). Further, the amount of analyte in the initial sample may be determined by comparing the generated electrochemical signal from the analyte with signals determined from known levels of the same analyte.

Many factors may cause the signal detected from identical samples to vary from sensor to sensor. For example, the extent of surface oxidation of the electrode, fouling, the quality of working electrode fabrication (in particular in connection with nanosensors), the operating conditions, and other factors may cause variability. In some embodiments that use a mediator, the mediator may provide an internal baseline signal that may be used to normalize signals obtained from different sensors. For example, if the signal from the mediator at a particular concentration is low relative to an expected value, the guanine signal may be proportionally low as well. Thus, corrections may be made to account for the low sensitivity of the particular sensor. Additionally or alternatively, signals from different sensors may be compared by plotting (1) the ratio of the signal from the analyte (scan 1 minus scan 2) to the signal of the analyte plus the mediator (scan 1) versus (2) mediator concentration.

In some embodiments, multiple unique sets of complementary oligonucleotides can be used at the same time. For example, in addition to the first plurality of oligonucleotides that are appended to a non-magnetic bead and the second plurality of oligonucleotides that are immobilized on an electrode, an embodiment may further comprise a third plurality of oligonucleotides that are appended to a non-magnetic bead and a fourth plurality of oligonucleotides (complementary to the third plurality of oligonucleotides) that are immobilized on an electrode that differs from the electrode to which the second plurality of oligonucleotides are immobilized. In such embodiments, the third/fourth plurality of oligonucleotides may be allocated for specific analytes, thereby permitting the measurement of multiple analytes from the same sample. Further, a unique set of complementary oligonucleotides may be used as a control to ensure the proper functioning of upstream steps. In other words, a known concentration of a control analyte may be added to the sample and detection of that analyte may be used as a positive control for upstream steps. In some embodiments, more than two analytes are detected and/or measured. For example, in some embodiments, more than 3, 5, 10, 20, or 100 analytes are detected simultaneously (or nearly simultaneously).

Detection methods, techniques, and systems described herein may possess one or more advantages over known detection/quantification techniques and systems. For example, the techniques disclosed herein may allow for the detection of analytes at lower concentrations than other techniques. Further, the detection systems disclosed herein may allow for the detection of analytes without requiring the use of optically detectable tags, such as those commonly used in a traditional enzyme-linked immunosorbent assay (“ELISA”). Such methods, techniques, and systems may allow for the detection of analytes more rapidly, easily, and/or inexpensively than other techniques.

To further illustrate these embodiments, the following examples are provided. These examples are not intended to limit the scope of the claimed invention.

### Example 1

### A—Apparatus and Reagents

Graphene oxide deposition and differential pulse voltammetry (“DPV”) were carried out using a Gamry Reference 600 potentiostat (Gamry Instruments, Warminster, Pa., USA). These graphene oxide deposition and DPV experiments used a conventional three-electrode system, which consisted of a bare or modified glassy carbon electrode (“GCE”) (3 mm diameter; BASi, West Lafayette, Ind., USA; Cat. No. MF-2012) and a platinum mesh as a counter-electrode.

Graphene oxide was purchased from Graphene Supermarket (Calverton, N.Y., USA). E. coli O157:H7 nonpathogenic strain was obtained from ATCC (Cat. #700728). The E. coli O157:H7 antibody-coated magnetic beads for pathogen extraction (Dynabeads MAX E. coli 0157 kit) were obtained from Invitrogen (Carlsbad, Calif., USA). Streptavidin-coated polystyrene beads were purchased from Bangs Laboratories Inc. (Fishers, Ind., USA; Cat. No. CP01N). Biotin-labeled BacTrace anti-E. coli O157:H7 antibody was purchased from Kikegaard and Perry Laboratories (KPL Inc., Gaithersburg, Md., USA; Cat. No. 16-95-90). Sulfo-NHS (N-hydroxysulfo-succinimide) and EDC (1-ethyl-3(3-dimethyl aminopropyl carbodiimide hydrochloride) were obtained from Pierce/Thermo Fisher (Rockford, Ill., USA). Sodium hydroxide was ordered from Macron Fine Chemicals (Center Valley, Pa., USA). Tris(2,2′-bipyridyl)ruthenium(II) chloride hexahydrate (Ru(bpy)₃Cl₂) was purchased from Sigma Aldrich (St. Louis, Mo., USA; Cat. No. 224758-1G). The oligonucleotides were obtained from the DNA-Peptide core facility at the University of Utah (Salt Lake City, Utah, USA).

All reagents were of analytical grade and were used as received without further purification. Unless otherwise specified, ultra-pure deionized water prepared by Purelab System (ELGA, Purelab, UK) was used throughout the experiment.

### B—Culturing of E. coli O157:H7

Following protocols supplied by ATCC (2014), a freeze-dried pellet of E. coli O157:H7 from ATCC was hydrated by adding 1 mL Difco Nutrient Broth (Becton Dickinson, Cat. No. 234000). The hydrated sample was then placed in an additional 5 mL of Difco Nutrient Broth. A 200 μL sample from this broth was placed on an agar plate prepared using Difco Nutrient Agar (Becton Dickenson, Sparks, Md., USA; Cat. No. 213000). The broth and agar plate were incubated at 37° C. for 36 h. After incubation, the broth culture was preserved using a protocol supplied by ATCC. The culture broth was centrifuged at 1000×g for 10 min to compact the bacteria into a pellet. The supernatant broth was poured off and 3 mL of fresh broth was added to the pellet. Then, 3 mL of sterilized 20% glycerol (v/v) was added to the culture. The culture was then placed in Nalgene Cryogenic vials (Thermo Scientific, Rockford Ill., USA) and stored at −135° C.

To seed E. coli in PBS buffer, the stored E. coli O157:H7 was plated on agar plates for 16 h. The E. coli grown on the plate were then collected using a sterile pipette tip and placed in 10 mL of 1×PBS. After vortexing this mixture, the concentration of E. coli was diluted to achieve an O.D. 600 of 0.1 (corresponding to a concentration of approximately 50 million bacteria/mL). Then 100 μL of this solution was serially diluted in 1×PBS to achieve different concentrations of 100 mL samples. The final concentration of E. coli O157:H7 was confirmed by plate counting.

### C—Pre-Concentration of E. coli O157:H7 from Seeded PBS Buffer Sample

PBS buffer samples (100 mL) that were seeded with E. coli O157:H7 as described above were concentrated into 1 mL samples as described below. The 100 mL sample was placed in a custom filtration device that was attached to a vacuum flask. The pressure within the vacuum flask was reduced to −55 kPa to pull the liquid through a 0.1 μm Durapore membrane filter (Millipore, Billerica, Mass., USA; Cat. No. VVLP04700), thereby trapping bacteria and solids larger than 0.1 μm. The filter was then removed from the device and inserted into a 1.5 mL Eppendorf tube containing 1 mL of 1×PBS. The sample was then vortexed for a minute to free the bound bacteria. The filter was subsequently removed from the Eppendorf tube. The efficiency of E. coli O157:H7 capture was determined by plating and incubating pre-concentrated and concentrated samples at 37° C. for 12 h and comparing the resulting colonies.

### D—Immunomagnetic Separation of E. coli O157:H7

Magnetic beads that were attached to antibodies with specificity to E. coli O157:H7 (Dynabeads) were used to isolate E. coli O157:H7. Briefly, the magnetic beads (20 μL) were added to the 1 mL samples obtained above. The tubes containing these 1 mL samples and the magnetic beads were then placed on a Mini-Lab Roller rotating mixer (Labnet International Inc., Edison, N.J., USA) and rotated at 24 rpms for 10 min. The tubes were then inserted into a custom-built magnetic capture unit for 3 minutes, occasionally inverting the tubes to facilitate concentration of the beads into a pellet. A portion of the supernatant solution (100 μL) was then pipetted onto an agar plate to test for any E. coli O157:H7 not captured by the beads. The rest of the supernatant was removed without disturbing the pellet. After removing the tube from the magnetic capture unit, 1 mL of 1× Dynabeads wash buffer was added to the tube, and the tube was then returned to the rotating mixer for 3 minutes. The process of mixing, plating 100 μL of supernatant, removing the remaining supernatant, and washing with 1 mL of 1× buffer was repeated two more times for a total of three wash cycles. After removing the remaining supernatant of the final wash, 100 μL of 1× buffer was added to and briefly mixed with the magnetic beads. A known amount of the resulting mixture was then plated on a final agar plate. The plates were all incubated at 37° C. for 12 h before colonies were counted to test the efficiency of the magnetic bead extraction process.

The average percent recovery of E. coli from the samples was 47%. This was a substantial increase in percent recovery relative to recovery from unconcentrated samples (22%). Due to the use of E. coli-specific antibodies, recovery was quite specific for E. coli O157:H7. Samples that initially included 3000 colony-forming units (“CFUs”) salmonella yielded, on average, a 0.4% extraction efficiency, while the extraction efficiency of E. coli O157:H7 was 95%

### E—Attachment of Antibody and Guanine-Rich Oligonucleotides to Streptavidin-Coated Polystyrene Beads

Biotinylated anti-E. coli O157:H7 antibodies (12.5 μL of 1 mg/mL solution) were added to 20 μL of streptavidin-coated non-magnetic polystyrene beads to couple the antibodies to the beads. Subsequently, biotinylated poly-G (GGGGGGGGGGGGGGGGGGGG/3′-biotin) (2.5 μL of a 50 μM solution) was also added to the same streptavidin-coated non-magnetic polystyrene beads.

### F—Attachment of Non-Magnetic Polystyrene Beads to Magnetic Bead-E. coli O157:H7 Complexes

To bind the non-magnetic polystyrene beads to the magnetic bead-E. coli O157:H7 complex, a 20 μL aliquot of the functionalized polystyrene beads (1% solids) was added to 20 μL of the mixture of resuspended magnetic beads and captured E. coli O157:H7 that was produced as described in Example 1d. The resulting mixture was mixed by pipet every 5-7 minutes over a 20-minute period.

### G—Preparation of the Electrode

Graphene oxide (25 mg) was added to 50 mL of 1×PBS. The resulting mixture of graphene oxide was exfoliated by ultra-sonication for 30 minutes to form a homogeneous brown colloidal dispersion with a concentration of 0.5 mg/mL. The graphene oxide in the colloidal dispersion was electrodeposited onto a glassy carbon electrode using a procedure similar to that reported in Chen et al., 13 Electrochemistry Communications 133-37. The glassy carbon electrodes were polished with an alumina slurry (with alumina particles of 0.5 μm) and sonicated in anhydrous ethanol and deonized water prior to electrodeposition. The cyclic voltammetric reduction was performed in the graphene oxide mixture under magnetic stirring, using a three-electrode system. The cyclic voltammeter was run from a potential of 1 to −1.5 Vat a scan rate of 50 mV/s for 18 cycles. Subsequently, the reduced graphene oxide-glassy carbon electrode (RGO-GCE) was washed with deionized water and dried in a stream of nitrogen.

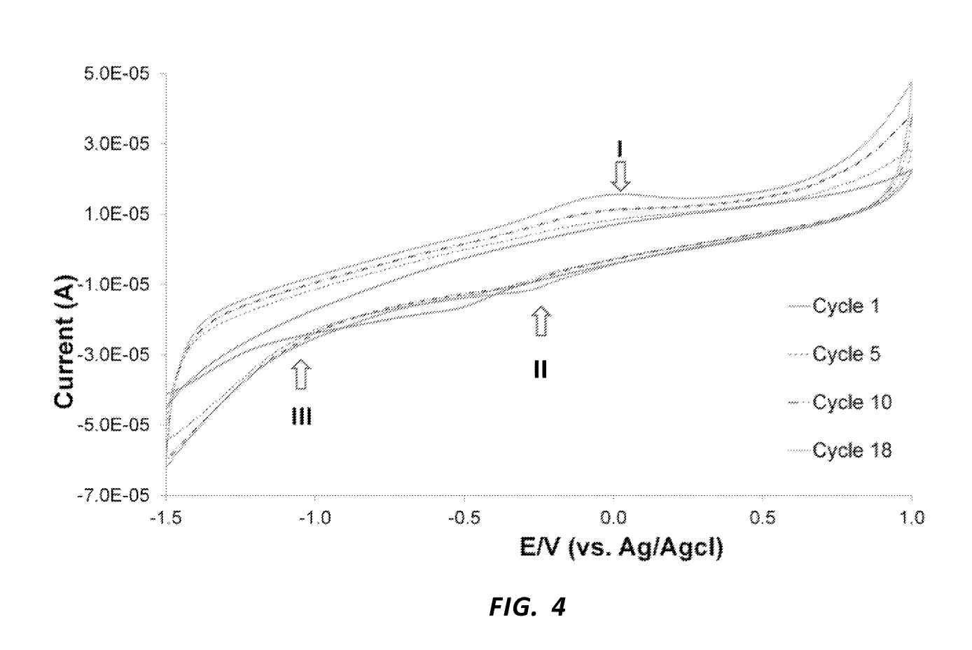

FIG. 4 is a graph depicting cyclic voltammetry data from the deposition of graphene oxide on the glassy carbon electrode surface. The graph shows one anodic peak (I) and two cathodic peaks (II and III). The cathodic peak III is attributed to the electrochemical reduction of graphene oxide, and the anodic peak I and the cathodic peak II are attributed to a redox pair of some electrochemically active oxygen-containing groups on the graphene plane that are too stable to be reduced by cyclic voltammetry under the conditions used here. Peak current generally increases with successive scans, indicating the deposition of reduced graphene oxide on the glassy carbon electrode surface. Electrodeposition of the graphene occurs only on conducting surfaces. The resultant graphene coating is quite stable due to its poor solubility in common solvents.

### H—Functionalization of Oligonucleotide Probes on the Electrode and Hybridization of Oligonucleotide Targets to the Probes

To functionalize the reduced graphene-oxide glassy carbon electrode (RGO-GCE), the RGO-GCE was activated by etching it with 1 M NaOH at 1.5 V to create carboxylic acid functional groups on the electrodeposited graphene oxide, as described in Kim et al., Microfluidic Integrated Multi-Walled Carbon Nanotube (MWCNT) Sensor for Electrochemical Nucleic Acid Concentration Measurement, 185 Sensors and Actuators B: Chemical 370-76, which is hereby incorporated by reference in its entirety. To convert the carboxyl groups to amine-reactive NHS esters, 10 μL of 100 mM sulfo-N-hydroxy succinimide, 400 mM EDC in 0.1 M MES buffer (pH=5.9) was pipetted onto the RGO-GCE surface and incubated for 1 h. The electrode surface was then washed with MES buffer. Subsequently, 10 μL of 25 μM amine-terminated cytosine probes (CCCCCCCCCCCCCCCCCCCC/3′-NH₂) was pipetted onto the activated RGO-GCE surface and incubated for 1 h. The surface was then washed with 1×PBS to wash off unattached cytosine-rich probes. Finally, the solution comprising the magnetic bead-E. coli-secondary bead complex was added to the probe-RGO-GCE for 1 h. The electrode surface was subsequently washed with 1×PBS before electrochemical detection.

### I—Fluorescent Microscopy Characterization of Probe-Target Hybridization

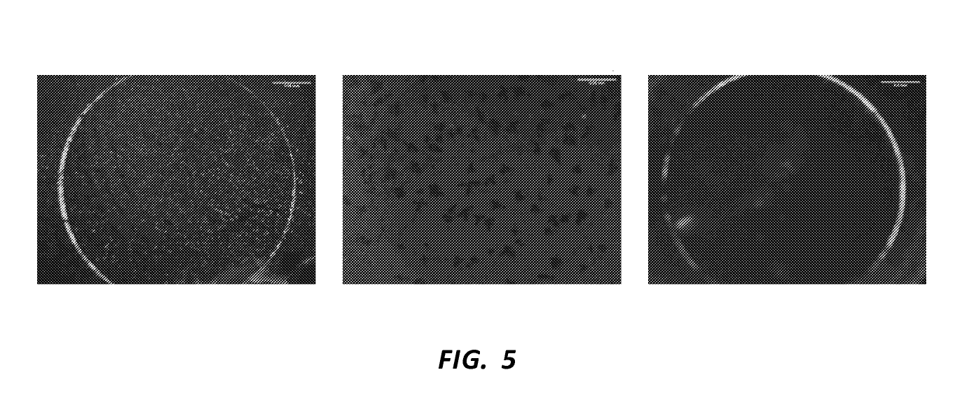

To confirm hybridization of the C-rich oligonucleotide probe to the G-rich oligonucleotide target, LCGreen intercalating dye (2 μL) was added to hybridized oligonucleotides that were immobilized on the electrode surface, and the system was visualized by fluorescence microscopy (4×, 500 millisecond exposure, Olympus IX81 inverted microscope, Olympus DP71 12-bit CCD color camera, FITC filter). The images were analyzed using Olympus DP Controller imaging software (Melville, N.Y., USA). FIG. 5 provides fluorescence images of three samples. The first sample (left) was processed as described above in the presence of analyte (200 cfu in 100 mL). The second sample (middle) and the third sample (right) served as negative controls. These samples were processed like the first sample, but included either no analyte (second sample) or no bead bound with G-rich oligonucleotides (third sample). The image for the first sample shows that a significant number of G-rich oligonucleotides are bound to C-rich oligonucleotides that have been immobilized on the electrode surface.

### J—Detection Via Amperometry (Differential Pulse Voltammetry)

Initially, a baseline differential pulse voltammetry curve was established by obtaining measurements of an RGO-GCE electrode with only probes (i.e., C-rich oligonucleotides) attached. Target (G-rich) oligonucleotides from samples containing different concentrations of E. coli (0, 3, 20, 200, 300 CFUs) were then hybridized to the cytosine-rich probes. Five consecutive differential pulse voltammetry scans were performed to determine the guanine oxidation peak corresponding to each of the hybridized targets. The differential value (S1-S5) was plotted for each target concentration (S1: first; S5: fifth scan). The differential pulse voltammetry measurements were conducted from 0.5 to 1.2 V (versus Ag/AgCl) in 0.2 M acetate buffer solution (pH 5) containing 5 μM Ru(bpy)₃²⁺ as the supporting electrolyte.

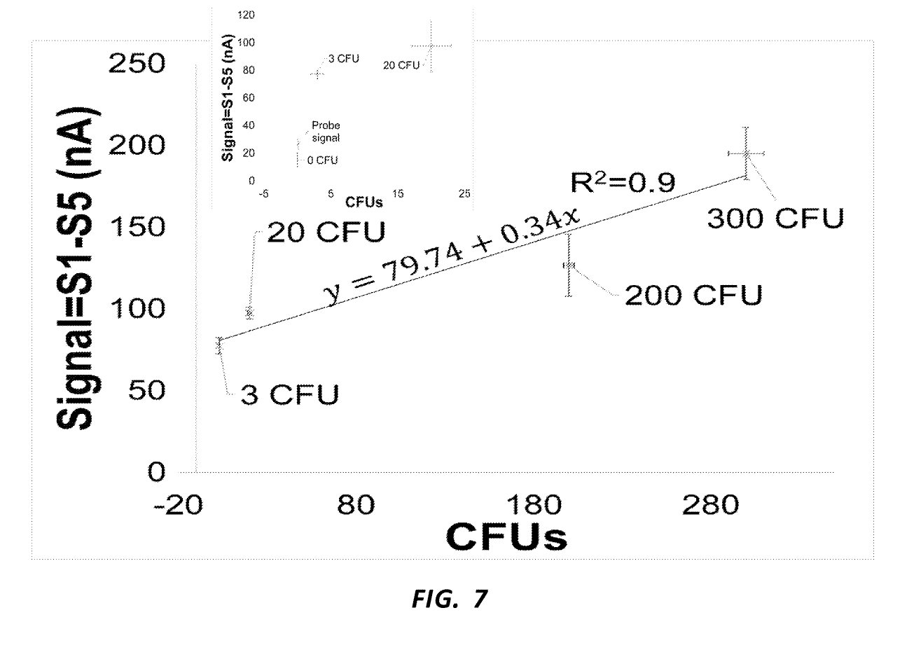

FIG. 7 shows the change in differential pulse voltammetry signals (S1-S5) as the amount of CFUs in the initial sample is varied from 0 CFUs to 300 CFUs. Signal strength increased as the amount of E. coli in the sample increased. The calibration curve shown in FIG. 7 is linear in the range from 3 CFUs to 300 CFUs, with a best-fit line of y=79.74+0.34x and an R² value of 0.9. The detection limit was 3 CFUs/100 mL with a signal-to-noise ratio of 3. A sample with 0 CFUs gave a signal of 15 nA, which corresponds to the baseline signal due to ruthenium electrolyte. See inset on FIG. 7. The average probe-only signal (RGO-GCE that has been functionalized with cytosine-rich probes) was slightly higher than the signal corresponding to 0 CFU. This difference is likely due to passivation of acetate in the electrolyte buffer during cycles of differential pulse voltammetry.

### Example 2

### Detection of E. Coli O157:H7 in Waste Water

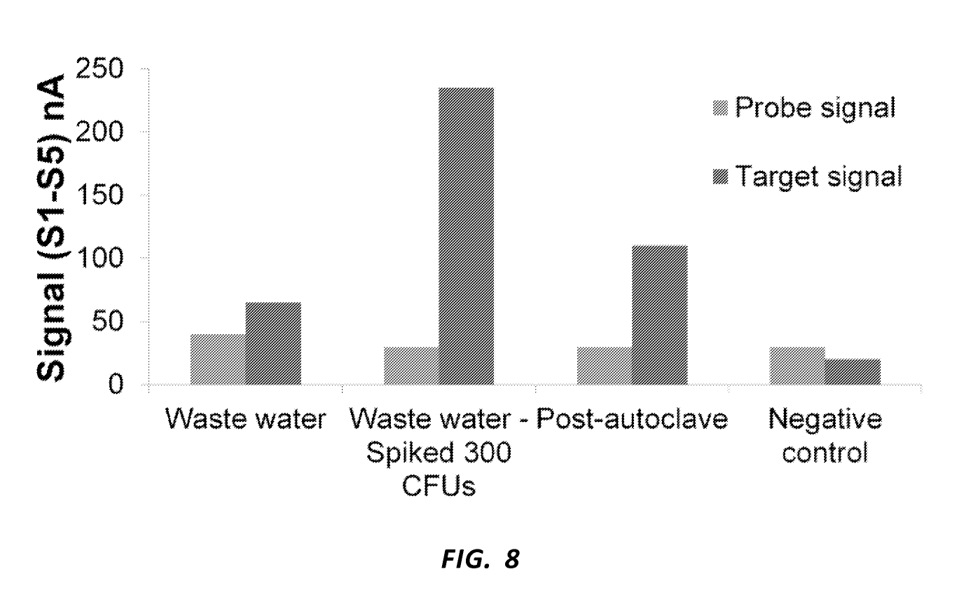

Waste water plant effluent was obtained from the Central Water Reclamation Facility (Salt Lake City, Utah, USA) and divided into three 100 mL samples. Two of the three waste water samples were seeded with 300 CFUs of E. coli O157:H7. One of these seeded samples was autoclaved before processing. Nothing was initially added to the non-seeded sample. Initially, vacuum filtration was employed using a 30 μm nylon net filter (Millipore, Billerica, Mass., USA; NY3004700) to remove any solids larger than 30 μm. Subsequently, each of the samples was concentrated to 1 mL using vacuum filtration as described in connection with Example 1. Deionized water (1 mL) was used as a negative control sample. The four samples were then processed (e.g., immunoseparation, electrochemical detection) in a manner similar to that described in Example 1 to determine the concentration of E. coli O157:H7 in each sample. FIG. 8 is bar graph depicting the change in signal magnitude (S1-S5) for each sample.

### Example 3

### A—Conjugation of Guanine-Rich Oligonucleotide to Nonmagnetic Beads

Guanine-rich oligonucleotides (GTGGGTGGGTAAGGAGTGAGGGTGG GAGTT) were conjugated to a nonmagnetic bead (15.28 μm) at the maximum packing density for the bead (˜10¹²/cm²) (7.3×10⁶ oligonucleotides/bead) in a manner similar to that described above in connection with Example 1. Because each oligonucleotide included 20 guanine bases, each bead was conjugated to 1.46×10⁸ guanine bases.

### B—Immunomagnetic Separation of E. coli O157:H7 from Water Samples

Magnetic beads that were attached to antibodies with specificity to E. coli O157:H7 (Dynabeads) were used to isolate E. coli O157:H7. Briefly, samples (1 mL) with known, low-level concentrations of E. coli O157:H7 (5×10⁻²¹ to 5×10⁻¹⁹) were exposed to approximately 2 million magnetic beads that were conjugated with antibodies specific for E. coli O157:H7. Immunomagnetic separation was used to separate the E. coli from other components within the sample.

### C—Binding of Non-Magnetic Beads to Isolated E. coli O157:H7

After repeatedly washing and separating the E. coli by immunomagnetic separation, the magnetic beads were released from the magnetic field and mixed with a solution containing 10,000 nonmagnetic beads that were conjugated to a second antibody specific to E. coli O157:H7. Each non-magnetic bead was also conjugated with guanine-rich oligonucleotide tags as described above such that each bead was conjugated to approximately 1.46×10⁸ guanine nucleobases. The recovery rate of E. coli O157:H7 from the sample after this step was estimated to be 60% by plating and incubating samples before and after the immunoseparation step.

### D—Conjugation of C-Rich Oligonucleotides to a Glassy Carbon Electrode

A graphene oxide nanostructure was formed on a glassy carbon electrode (GO-GCE) and functionalized with a cytosine-rich oligonucleotide (CACCCACCCATTCCTCACTCCCACCCTCAA-3′ amine) that is complementary to the G-rich oligonucleotide (GTGGGTGGGTAAGGAGTGAGGGTGGGAGTT) that is conjugated to the nonmagnetic beads. The cytosine-rich oligonucleotides were immobilized onto the 1 mm² electrode at maximum packing density (1×10¹⁰ oligonucleotides/mm²). Thus, the number of cytosine-rich oligonucleotides on the electrode (1×10¹⁰) exceeded the number of guanine-rich oligonucleotide tags to be immobilized through hybridization onto the surface. The number of C-rich probes on the electrode is an indicator of the dynamic range of analyte concentrations that may be measured using the electrode.

### E—Hybridization and Electrochemical Detection

Approximately 1.3×10⁷ of the G-rich oligonucleotide tags (2.6×10⁸ guanine nucleobases) were eluted from magnetically immobilized sandwich structures and delivered to the RGO-GCE which was conjugated with approximately 1×10¹⁰ C-rich oligonucleotide probes to form approximately 1.3×10⁷ duplexes. With the electrode in a buffer of 0.2 M NaOAc (pH=5), 5.0 μM Ru(bpy)₃²⁺, differential pulse voltammetry was used to investigate the extent of guanine oxidation. The three-electrode system included a platinum counter electrode and a Ag/AgCl reference electrode.

The detection signal peak generated in the first scan from guanine and Ru(bpy)₃ was approximately 80 nA. The detection signal generated in subsequent scans was approximately 25 nA, with a maximum variability between such scans of approximately 10 nA. In other words, the difference between the signal generated from the first scan and the signal from subsequent scans was approximately 55 nA. Because the signal detected from the first scan exceeded baseline levels after accounting for variability, the presence of E. coli O157:H7 in the sample was confirmed.

### Example 4

### Investigation of Dynamic Range, Generation of a Calibration Curve, and Determination of Analyte Concentration

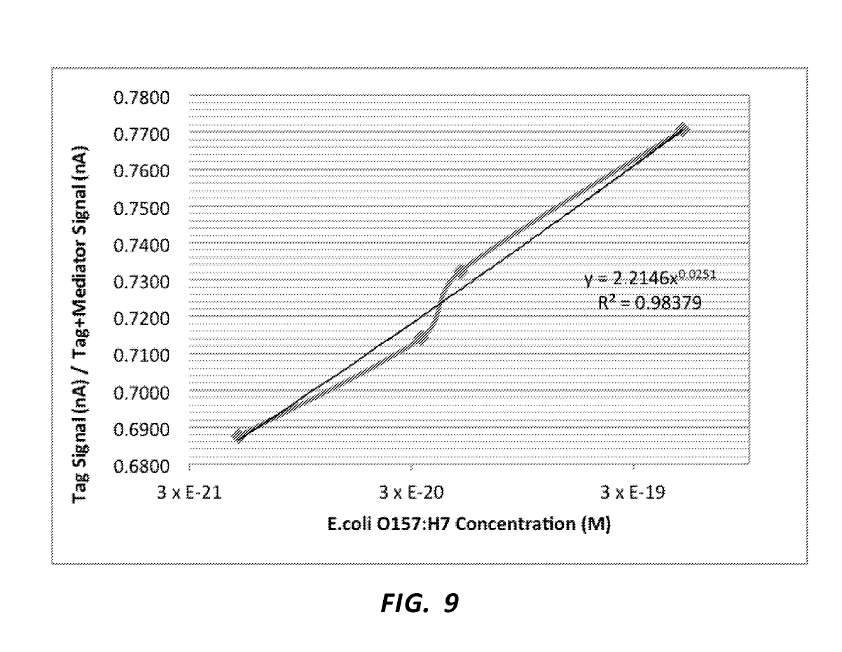

The limits of the dynamic range for techniques similar to those described above was investigated by determining the electrochemical signal generated from samples that had very low levels of E. coli O157:H7 concentrations (5×10⁻²¹ M and 5×10⁻¹⁹ M). The samples were analyzed substantially as described in Example 1. FIG. 9 provides a graph that plots normalized signal (y-axis) versus concentration of E. coli (x-axis). The signal was normalized by subtracting off baseline levels (29.2±4.2 nA) corresponding to the oxidation of Ru(bpy)₃. A simple linear regression is depicted in FIG. 9, with an R² value of 0.984. Because the signal response within this range was substantially linear, the linear regression was used as a calibration curve to determine the concentration of unknown analytes within this range.

### Example 5

### Manufacture of Graphene Chip Electrodes

An electrode comprising a silicon wafer that is suitable for use in electrochemical detection was manufactured as set forth below.

A 100 nm silicon dioxide layer was grown on a 4 inch P-type silicon wafer using wet oxidation. Subsequently, an adhesion promoter, hexamethyldisilazane (HMDS) was coated on the silicon wafer using a YES1 HMDS oven. A 20 micron layer of AZ9260 was spin-coated on the wafer at 1000 RPM for 30 seconds and baked on a hotplate at 110° C. for 5 minutes. The electrode patterns (bond and detection pads) were exposed using UV light and developed on the AZ9360 coated wafer using conventional photolithography. The developed wafer was dried overnight in a clean room hood. Subsequently, they were diced and pyrolyzed at 1000° C. in a tube furnace (Nitrogen atmosphere). The thickness of the photoresist after pyrolysis was reduced to 4 microns.

Graphene oxide was deposited on the detection pads of the pyrolyzed electrode by drop casting. In particular, 1 μL of 2-5 mg/mL carboxyl acid functional graphene oxide (GO-COOH) was dip coated on top of the pyrolyzed electrode and allowed to dry for one hour.

To convert the carboxyl groups on the electrode to amine-reactive NHS esters for attachment to amine-terminated probes, 1 μL of freshly prepared 100 mM Sulfo-NHS and 400 mM EDC in 0.1 M of MES buffer (pH=5.9) was pipetted on the pyrolyzed electrode surface for 1 hour and then washed with MES buffer. Subsequently, 1 μL of 25 μM cytosine probes in 1× PBS was pipetted on the activated pyrolyzed electrode surface for 1 hour, followed by washing with 1×PBS to wash off the excess unattached cytosine probes. Finally, the hybridization reactions were performed by incubating the target (magnetic bead/E. coli/secondary bead complexes) solution on the probe-pyrolyzed electrode for 1 hour. The electrode surface was subsequently washed with 1×PBS before electrochemical detection.

### Example 6

### Measurement Capabilities Relative to Other Techniques

Table 1 compares the measurement capabilities of different biosensors. This table shows the increased sensitivity of guanine-nucleobase amplification techniques relative to other techniques. The values and estimates of the sensitivity of other techniques are sourced from references that describe detection limits for a wide range of similar groups of technologies and platforms. Thus, the listed technologies can have values that deviate somewhat from the numbers set forth in Table 1. The term “measurement capabilities” is a general term that encompasses the concepts of sensitivity, limit of detection, and limit of quantification.

TABLE 1

| | | | | | | | | | 8 |
|---|---|---|---|---|---|---|---|---|---|
| | | | 2 | 3 | | | 6 | 7 | Electrochemical |
| | | 1 | Glucose | Glucose | 4 | 5 | Bead | Immuno- | Detection via |
| | Measurement | Blood | Enzyme | Nano- | Direct | Sandwich | Sandwich | nano- | Guanine |
| | Capabilities | Glucose | Biosensor | sensor | ELISA | ELISA | ELISA | sensor | Amplification |
| 1 | Detectable | 1.1 × 10⁻³ | 3.3 × 10⁻⁵ | 1.0 × 10⁻⁷ | 1.3 × 10⁻¹⁰ | 8.3 × 10⁻¹⁴ | 7.6 × 10⁻¹⁷ | 1.7 × 10⁻¹³ | 5.0 × 10⁻²¹ |
| | Concentration | | | | | | | | |
| | (M) | | | | | | | | |
| 2 | Sample Volume | 3 | 3 | 3 | 100 | 100 | 25 | 100 | 1000 |
| | (μL) | | | | | | | | |
| 3 | # of Analytes in | 2.0 × 10¹⁴ | 6.0 × 10¹² | 1.8 × 10¹⁰ | 7.5 × 10⁹ | 5.0 × 10⁶ | 1.1 × 10³ | 1.0 × 10⁷ | 3 |
| | Sample | | | | | | | | |
| 4 | Detectable | 1 | 1 | 1 | 1 | 200 | 1 | 3000 | 1.5 × 10⁸ |
| | Tags/Analyte | | | | | | | | |
| 5 | Recovery by | 100 | 100 | 100 | 80 | 60 | 100 | 60 | 60 |
| | Antibodies (%) | | | | | | | | |
| 6 | Detectable | 2.0 × 10¹⁴ | 6.0 × 10¹² | 1.8 × 10¹⁰ | 6.0 × 10⁹ | 6.0 × 10⁸ | 1.1 × 10³ | 1.8 × 10¹⁰ | 2.6 × 10⁸ |
| | Targets | | | | | | | | |

In Table 1, the first row shows the lowest concentration of analyte that may be measured with the biosensor, while the second row shows typical sample volume. The remaining rows show the number of total analytes in the sample, the number of detectable tags per analyte, the percent of analyte recovered through interaction with antibodies, and the number of detectable targets in the sample.

As can be deduced from Table 1, common methods of determining blood glucose levels (see columns 1-3) are generally much less sensitive than the guanine-amplification techniques disclosed herein. For example, a commercially available blood glucose meter (Abbott FreeStyle, Abbott Diagnostics Care, Alameda, Calif., USA) can detect 3.3×10⁻¹° moles (2×10¹⁴ molecules) of glucose from a 0.3 μL sample (1.1 mM). See column 1. A glucose enzyme biosensor (Accu Check Compact Plus portable instrument, Roche Diagnostics GMBH, Mannheim, Germany) can detect 0.33 mM glucose in a 0.3 μL sample (6.0×10¹² glucose molecules). See column 2. A glucose nanosensor has been shown to have lower detection limits than those of the Abbott FreeStyle glucose meter or the Accu Check Compact Plus. Zhu reports a measurement capability of approximately 0.0001 mM for certain nanosensors, which corresponds to the detection of approximately 1.8×10¹⁰ molecules. Zhu et al., Detection of E. coli O157:H7 by immunomagnetic separation coupled with fluorescence immunoassay, 30 Biosensors and Bioelectronics 337-341 (2011). Although the glucose nanosensor disclosed in Zhu shows improved sensitivity relative to other glucose biosensors, this nanosensor is not capable of detecting analytes at very low concentrations (e.g., 5.0×10⁻¹⁸ mM in a 1 mL sample), such as the guanine amplification techniques described herein. See column 8. Further, many glucose nanosensors are not commercially viable due to high fabrication cost, signal inconsistency from sensor to sensor, inconsistent fabrication quality, and difficulties in measuring low signal due to ambient noise.

Columns 4 and 5 of Table 1 provide relative measurement capabilities of representative direct and sandwich ELISAs used for the detection of proteins. The values and estimates are provided from ELISA technical documents published by KPL Inc. (Gaithersburg, Md., USA) and Thermo Scientific (Rockford, Ill., USA).

For typical ELISAs using horseradish peroxidase and colorimetric detection, the detection limit of an analyte (e.g., Interleukin 2) is approximately 2,125 pg/mL (125 pM) for direct ELISA assays and 1.4 pg/mL (0.08 pM) for sandwich ELISA assays. See columns 4 and 5. The increased detection limits for sandwich ELISA assays is due, at least in part, to the signal amplification.

Techniques, such as those disclosed here, involving guanine amplification and nanosensor detection provide many orders of magnitude greater amplification than that found in traditional ELISA assays. For example, for each analyte, up to 10¹⁵ electrochemically detectable tags (e.g., guanine nucleobases) may be measured. For example, guanine nucleobase detection was used to detect concentration levels of an analyte (i.e., E. coli) that are seven orders of magnitude lower than the lower detection limits for a sandwich ELISA as shown in Table 2.

Columns 6 and 7 of Table 1 provide the relative measurement capabilities of emerging biodetection technologies. Bead sandwich ELISAs are similar to traditional ELISA assays, but the capture antibody is conjugated to a bead instead of to a solid substrate. The concentration is typically determined via detection of an optical label. Column 6 of Table 1 shows the lowest concentration of detected analyte as reported by Quanterix. While such systems may provide a substantial increase in detection capabilities relative to traditional sandwich ELISA techniques, none have the amplification capability of 10¹⁵ electrochemically detectable tags per analyte, which facilitates the detection of very low levels of analyte.

Another emerging biodetection technology, in addition to bead sandwich ELISA assays, involves the use of immunonanosensors. See column 7. Immunonanosensors that use biosensors to electrochemically detect proteins have been shown to detect analytes at a concentration of approximately 0.17 pM. See Chikkaveeraiah et al., Electrochemical Immunosensors for Detection of Cancer Protein Biomarkers, 28 ACS Nano 6546-61 (2012).

As shown in Table 1, none of the techniques disclosed in columns 1-7 is capable of detecting an analyte at concentrations as low as those detected herein using guanine nucleobase amplification (5×10⁻²¹ M). See column 8. Thus, guanine nucleobase amplification techniques may have greater sensitivity, lower detection limits, and/or lower quantification limits than other electrochemical techniques. For example, analyte at a concentration of 5.0×10⁻¹⁸ mM in 1 mL of sample may be detected and quantified. Column 8, rows 1 and 2.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure, that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

1. A method of detecting an analyte in a sample, the method comprising:
(A) adding a first analyte-binding agent to the sample to form a first complex that comprises the first analyte-binding agent and the analyte;
(B) adding a second analyte-binding agent to the sample to form a second complex comprising the second analyte-binding agent and the analyte, wherein the second analyte-binding agent is coupled to a plurality of first oligonucleotides comprising a first sequence;
(C) removing an analyte-depleted portion from the sample;
(D) binding the plurality of first oligonucleotides to a plurality of second oligonucleotides comprising a second sequence that is substantially complementary to the first sequence, wherein the plurality of second oligonucleotides is immobilized on an electrode; and
(E) electrochemically detecting oxidation of one or more nucleobases of the plurality of first oligonucleotides.

2. The method of claim 1, wherein the electrode comprises graphene oxide.

3. The method of claim 1, wherein the first analyte-binding agent comprises an antibody that is conjugated to a magnetic particle.

4. The method of claim 1, wherein
the first oligonucleotide comprises one or more guanine nucleobases; and
electrochemically detecting oxidation of one or more nucleobases comprises detecting oxidation of the one or more guanine nucleobases.

5. The method of claim 1, wherein electrochemically detecting oxidation of one or more nucleobases of the plurality of first oligonucleotides comprises use of a mediator.

6-7. (canceled)

8. The method of claim 1, wherein the first analyte-binding agent comprises an antibody, the second analyte-binding agent comprises an antibody, and the first analyte-binding agent and the second analyte-binding agent bind to different epitopes of the analyte.

9-10. (canceled)

11. The method of claim 1, wherein the first oligonucleotide has a plurality of nucleobases, the majority of which are guanine nucleobases.

12. The method of claim 1, wherein electrochemically detecting oxidation of one or more nucleobases of the plurality of first oligonucleotides comprises the use of voltammetry.

13. The method of claim 1, wherein the second analyte-binding agent comprises a non-magnetic particle that is conjugated to more than about 10,000 oligonucleotides.

14. (canceled)

15. The method of claim 1, wherein
step C occurs after step B;
step D occurs after step C; and
step E occurs after step D.

16-21. (canceled)

22. The method of claim 1, wherein the analyte to be detected is present at an initial concentration of below 1×10⁻¹² M.

23-24. (canceled)

25. A method of detecting an analyte in a sample, the method comprising:
adding a first analyte-binding agent to the sample, the first analyte binding agent comprising a first analyte-specific antibody and a magnetic bead;
adding a second analyte-binding agent to the sample, the second analyte binding agent comprising a second analyte-specific antibody, a non-magnetic bead, and a plurality of first oligonucleotides comprising a first sequence;
binding the plurality of first oligonucleotides to a plurality of second oligonucleotides comprising a second sequence that is substantially complementary to the first sequence, wherein the plurality of second oligonucleotides is immobilized on an electrode; and
electrochemically detecting oxidation of one or more nucleobases of the plurality of first oligonucleotides.

26-29. (canceled)

30. The method of claim 25, wherein the first analyte-binding agent and the second analyte-binding agent each independently comprise one or more of an antibody, a peptide aptamer, and an oligonucleotide aptamer.

31-36. (canceled)

37. The method of claim 36, wherein the second analyte-binding agent comprises more than 10⁸ oligonucleotides.

38. The method of claim 25, wherein the second analyte-binding agent does not comprise a gold particle.

39-40. (canceled)

41. The method of claim 25, wherein the second analyte-binding agent is coupled to the plurality of first oligonucleotides via a linker.

42. (canceled)

43. A system for measuring the presence of an analyte in a sample, the system comprising:
a first analyte-binding agent configured to selectively bind to an analyte and facilitate separation the analyte from other components in a sample;
a second analyte-binding agent configured to bind to the analyte, the second analyte-binding agent comprising a first portion and a second portion, wherein the first portion of the second analyte-binding agent is configured to bind to the analyte and the second portion of the second analyte binding agent comprises a plurality of first oligonucleotides comprising a first sequence;
a second plurality of oligonucleotides comprising a second sequence that is complementary to the first sequence, wherein the second plurality of oligonucleotides is immobilized on an electrode; and
a device configured to electrochemically detect oxidation of the plurality of first oligonucleotides.

44. The system of claim 43, wherein the electrode comprises graphene oxide.

45. The system of claim 43, wherein the electrode comprises elemental carbon.

46. The system of claim 43, wherein the first sequence consists of a plurality of nucleobases, wherein more than 40% of the plurality of nucleobases are guanine.


SEQUENCE LISTING (4 sequences)

SEQ ID NO: 1 - DNA, 20 bp, Artificial Sequence
  Poly-G
gggggggggggggggggggg

SEQ ID NO: 2 - DNA, 20 bp, Artificial Sequence
  Amine-terminated cytosine probe
cccccccccccccccccccc

SEQ ID NO: 3 - DNA, 30 bp, Artificial Sequence
  Guanine-rich oligonucleotide
gtgggtgggtaaggagtgagggtgggagtt

SEQ ID NO: 4 - DNA, 30 bp, Artificial Sequence
  Cytosine-rich oligonucleotide
cacccacccattcctcactcccaccctcaa